SEQUENCE LISTING (5 sequences)

SEQ ID NO: 1 - protein, 458 aa, Homo sapiens
  MISC_FEATURE (152)..(152): Xaa at position 152 may be Glycine or Glutamate
  MISC_FEATURE (429)..(429): Xaa at position 429 may be Arginine or Histidine
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKXSDFASNCCSINSPPLYCDSEIDAELKNIL

SEQ ID NO: 2 - protein, 458 aa, Homo sapiens
  MISC_FEATURE (152)..(152): Xaa at position 152 may be Glycine or Glutamate
  MISC_FEATURE (429)..(429): Xaa at position 429 may be Arginine or Histidine
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPEATPTELAKLVNKXSDFASNCCSINSPPLYCDSEIDAELKNIL

SEQ ID NO: 3 - protein, 458 aa, Homo sapiens
  MISC_FEATURE (152)..(152): Xaa at position 152 may be Glycine or Glutamate
  MISC_FEATURE (429)..(429): Xaa at position 429 may be Arginine or Histidine
LERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYXQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPKELAKLVNKXSDFASNCCSINSPPLYCDSEIDAELKNIL

SEQ ID NO: 4 - protein, 83 aa, Artificial Sequence
  Synthetic peptide
  MISC_FEATURE (55)..(55): Xaa at position 55 can be any naturally occurring amino acid
LLKKELSSFIDKGQELCADYSENTFTEYKKKLAERLKAKLPDATPTELAKLVNKXSDFSNCCSINSPPLYCDSEIDAELKNIL

SEQ ID NO: 5 - protein, 84 aa, Artificial Sequence
  Synthetic peptide
  MISC_FEATURE (55)..(55): Xaa at position 55 can be any naturally occurring amino acid
LLKKELSSFIDKGQELCADYSENTFTEYKKKLAERLKAKLPDATPKELAKLVNKXSDFASNCCSINSPPLYCDSEIDAELKNIL

(19) United States
(12) Patent Application Publication
(10) Pub. No.: US 2018/0078613 A1
(43) Pub. Date: Mar. 22, 2018

(54) MACROPHAGE ACTIVATING FACTOR FOR TREATING BENIGN OR PRECANCEROUS PAPILLOMAS

(71) Applicant: EFRANAT LTD., Rehovot (IL)

(72) Inventors: Michal SHAHAR, Rishon Lezion (IL); Uri YOGEV, Yavne (IL); Rinat ROTEM-YEHUDAR, Tel-Aviv (IL)

(73) Assignee: EFRANAT LTD., Rehovot (IL)

(21) Appl. No.: 15/564,607

(22) PCT Filed: Apr. 6, 2016
(86) PCT No.: PCT/IL2016/050361

§ 371 Date: Oct. 5, 2017

Related U.S. Application Data
(60) Provisional application No. 62/143,827, filed on Apr. 7, 2015.

(51) Int. Cl. A61K 38/19; A61K 9/00; C07K 14/52; A61K 45/00

(52) U.S. Cl. CPC A61K 38/19; A61K 9/0019; C12N 2710/20011; A61K 45/05; C07K 14/52

(57) ABSTRACT

Provided are pharmaceutical compositions including macrophage activating factor derived from Gc protein (GcMAF) for use in treating benign or precancerous papillomas. Further provided are pharmaceutical compositions including GcMAF for use in treating recurrent respiratory papillomatosis (RRP).

### FIELD OF THE INVENTION

The present invention relates to pharmaceutical compositions comprising macrophage activating factor derived from Gc protein (GcMAF) for use in treating benign or precancerous papillomas. Particularly, the present invention relates to pharmaceutical compositions comprising GcMAF for use in treating recurrent respiratory papillomatosis (RRP).

### BACKGROUND OF THE INVENTION

Recurrent Respiratory Papillomatosis (RRP), also known as laryngeal papillomatosis, is a seriously debilitating disease. The disease is characterized by the growth of warts or benign lesions of the skin caused by hyperplasia and enlargement of contiguous dermal papillomas, predominantly present in the larynx and on the vocal cords, but can spread to the trachea and lungs. The respiratory papillomas can be deadly in juvenile RRP due to the small size of the upper airway in children. Lesions may grow very fast and death can result from airway obstruction. Traditionally, surgery is used to remove the affected tissue. However, surgical procedures in the airways involve considerable risk, particularly in children. Antiviral drugs are also used and the best success has been observed by injecting cidofotvir directly into the papillomas. However, direct injection into the papillomas of the lower airways is very complicated and requires special proficiency to avoid damage to the vocal cords and adjacent tissues. Regardless of the treatment used, the papillomas recur frequently, and therefore the patients usually undergo multiple surgical procedures and toxic systemic medications in an attempt to control their disease.

Papillomatosis also occurs in animals, e.g., domestic animals such as dogs and cats, where it is characterized by cauliflower-like skin and mouth lesions (also named oral papillomatosis). Typically, canine oral papillomatosis appears in young dogs below the age of two years whose immune system has not yet been fully matured or in old dogs whose immune system has been weakened. In young dogs, canine oral papillomatosis may be spontaneously regresses when the immune system matures. However, in moderate cases of canine oral papillomatosis, and particularly in severe cases of the disease where tens of lesions grow in the dog's mouth, these lesions are associated with excruciating pain and affect the ability of the dogs to eat and drink, and consequently the dogs lose their body weight. The medication imiquimod has been used to boost immune-mediated inflammation, and thus to facilitate destruction of the lesions. The antibiotic azithromycin has also been proven to help resolve oral lesions. However, in moderate to severe cases these medications are not always effective.

Recurrent Respiratory Papillomatosis (RRP) in humans and papillomatosis in domestic animals is caused by papilloma viruses, non-enveloped viruses having a double-stranded circular DNA. While infection with some subtypes of human papilloma virus (HPV) is recognized as one of the major causes of infection-related cancer worldwide, some HPV subtypes are not associated with malignant transformation. It is accepted that juvenile RRP is a perinatally-acquired infectious disease. It is caused by infection of newborns with mucosal tissue-tropic papillomaviruses, usually HPV-11 or HPV-6. This infection is usually transmitted to the nasopharynx of a newborn from the genital tract of its mother during vaginal delivery, although there is also evidence of transmission to the fetus in utero.

U.S. Pat. No. 6,797,491 discloses a method of treating recurrent respiratory papillomatosis which comprises administering, to a subject who has been identified as having recurrent respiratory papillomatosis, a composition comprising a fusion protein comprising an Hsp60 protein and a human papilloma virus (HPV) type 16 E7 protein or an antigenic fragment thereof.

U.S. Pat. No. 7,348,352 discloses compositions and methods that comprise the phytochemical Diindolylmethane, alone or in combination with immune potentiating steroids for treating common cutaneous warts (verrucae) and HPV related conditions of the oropharynx, larynx, genitalia, and uterine cervix.

U.S. Pat. No. 8,535,657 discloses stabilized liquid or freeze dried compositions comprising a mixture of synergistic proportions of recombinant gamma interferon and recombinant alpha interferon for the treatment of benign, non-physiological or malignant solid tumors in humans, among which laryngeal papillomatosis is listed.

U.S. Pat. No. 9,186,336 discloses methods of treating laryngeal recurrent respiratory papillomatosis (RRP) comprising locally administering to a phonatory mucosal surface in the larynx of the subject in need thereof an anti-VEGF antibody.

Macrophage Activating Factor Derived from Gc Protein

Macrophages are widely distributed immune cells that play an essential role in the innate and adaptive immune response to pathogens and in cancer cell destruction. Activation of macrophages which leads to their increased phagocytic and cytotoxic activity is mediated inter alia by macrophage activation factor derived from Gc protein.

Gc protein, also known as vitamin D-binding protein, is a glycoprotein of the α-2 macroglobulin fraction of human plasma having an apparent molecular weight of 52 kDa, constituting about 0.5% of plasma proteins in healthy human subjects. Two genes of Gc protein have been identified: Gc1 and Gc2 which differ from each other by four amino acids. Previous studies have indicated that Gc1 protein carries a trisaccharide composed of N-acetylgalactosamine attached to the core protein, sialic acid and galactose (the Gc1f and Gc1s* subtypes), or N-acetylgalactosamine, mannose and galactose (the Gels subtype) while Gc2 protein carries a disaccharide composed of N-acetylgalactosamine and galactose. It was described that the trisaccharides of the Gc1f and Gc1s* subtypes are hydrolyzed by β-galactosidase to yield a macrophage pro-activating factor which is further hydrolyzed by sialidase to yield the macrophage activating factor (GcMAF). The trisaccharides of the Gc1s subtype are hydrolyzed by β-galactosidase and mannosidase to yield the macrophage activating factor (GcMAF). The conversion of Gc2 to macrophage activating factor requires the cleavage of β-galactosidase only (Yamamoto et al., 1991, Proc. Natl. Acad. Sci. USA 88:8539-8543).

U.S. Pat. No. 5,177,002 discloses a process for producing a macrophage activating factor comprising contacting glycosylated mammalian vitamin D-binding protein in vitro with β-galactosidase, or β-galactosidase in combination with sialidase, α-mannosidase, or a mixture thereof, and obtaining the macrophage activating factor. U.S. Pat. No. 5,177,002 further discloses a method for inducing macrophage activation in an individual in need thereof comprising administering to the individual the human macrophage activating factor thus produced.

U.S. Pat. No. 6,410,269 discloses a process for cloning vitamin D-binding protein and its small domain (also known as domain III) in a baculovirus vector. The cloned Gc protein and the cloned domain III were treated with immobilized β-galactosidase and sialidase to yield macrophage activating factors, GcMAFc and CdMAF, respectively. U.S. Pat. No. 6,410,269 further discloses uses of the macrophage activating factors for the treatment of cancer, human immunodeficiency virus (HIV) infection and osteoporosis.

WO 2012/137199 discloses pharmaceutical compositions comprising Gc protein derived macrophage activating factor (GcMAF) essentially devoid of glycosidase enzymes and methods of use thereof for treating cancer or HIV-infected patients.

WO 2014/199373 to some of the inventors of the present invention discloses storage-stable pharmaceutical compositions comprising GcMAF and at least one pharmaceutically acceptable surfactant and/or a synthetic water-soluble polymer having surface activity and uses thereof for treating diseases associated with macrophage activation.

There remains an unmet need for improved methods of treating benign or precancerous papillomas, particularly of treating Recurrent Respiratory Papillomatosis (RRP), which methods are safe, do not involve surgical procedures, and which prevent recurrence of the disease.

### SUMMARY OF THE INVENTION

The present invention provides pharmaceutical compositions comprising macrophage activating factor derived from Gc protein (GcMAF) for use in treating benign or precancerous papillomas. In particular, the present invention provides pharmaceutical compositions comprising GcMAF for treating recurrent respiratory papillomatosis (RRP), specifically juvenile recurrent respiratory papillomatosis (JRRP).

The present invention is based in part on the unexpected discovery that macrophage activating factor derived from human Gc protein (GcMAF), when administered to dogs afflicted with oral papillomatosis was capable of eradicating the papillomas from the dog's mouth and face.

It is well accepted in veterinary medicine that in severe cases of canine papillomatosis, where conventional medications are ineffective in eradicating the disease, dogs should be anesthetized to death.

It is now disclosed for the first time that in a severe case of canine papillomatosis, where the dog's mouth was full of papillomas and therefore was unable to eat solid food and consequently lost weight, three weekly subcutaneous injections of a pharmaceutical composition comprising GcMAF arrested the disease progression and enabled the dog to start eating solid food. Additional weekly subcutaneous injections of the pharmaceutical composition comprising GcMAF for a period of about three months eliminated almost completely the papillomas from the dog's mouth and most of the papillomas from its face. Weekly injections of the pharmaceutical composition comprising GcMAF for two additional months resulted in complete disappearance of the papillomas from the dog's mouth and face, and the dog regained its normal weight. Not only that the skin and mouth lesions disappeared, the disease did not recur since termination of the treatment.

It is now further disclosed that in moderate cases of canine papillomatosis, only few administrations of a pharmaceutical composition comprising GcMAF were required to eradicate the papillomas from the dog's mouth and face. Thus, three to fifteen weekly administrations of the pharmaceutical composition comprising GcMAF were sufficient to achieve complete removal of the oral and facial lesions in dogs afflicted with moderate canine papillomatosis.

It is further disclosed that the pharmaceutical compositions comprising GcMAF of the present invention were safe, well tolerated, and did not cause any detectable adverse or side effects.

It is further disclosed that a pharmaceutical composition comprising GcMAF is effective in treating recurrent respiratory papillomatosis (RRP) in humans, and specifically moderate to severe juvenile RRP (JRRP). In addition, the pharmaceutical composition comprising GcMAF is shown to delay or even prevent recurrence of RRP, and specifically of JRRP. Thus, the pharmaceutical composition comprising GcMAF of the present invention is a highly efficacious medication of RRP, it fulfills a long-felt need for a therapy of moderate to severe RRP, particularly of moderate to severe JRRP, it elongates the intervals between debulking surgeries, and it can even eliminate the need of these surgeries.

According to one aspect, the present invention provides a pharmaceutical composition comprising macrophage activating factor derived from Gc protein denoted GcMAF, or a biologically active fragment thereof, and a pharmaceutically acceptable carrier for use in treating benign or precancerous papilloma.

According to some embodiments, the benign or precancerous papilloma is selected from the group consisting of laryngeal papilloma, known as recurrent respiratory papillomatosis (RRP), oral papilloma known as oral papillomatosis, conjuctival papilloma, verrucae plantares, verrucae vulgaris, anogenital warts (Condylomata acuminata, Condylomata plana, bowenoid papulosis), and focal epithelial hyperplasia. Each possibility represents a separate embodiment of the invention.

According to additional embodiments, the benign or precancerous papilloma is RRP. According to further embodiments, the RRP is pediatric or juvenile RRP (JRRP). According to yet further embodiments, the JRRP is selected from the group consisting of moderate JRRP and aggressive JRRP. According to a certain embodiment, the JRRP is aggressive JRRP. According to additional embodiments, the RRP is adult-onset RRP. According to additional embodiment, the adult-onset RRP is aggressive adult-onset RRP.

According to another embodiment, the benign or precancerous papilloma is anogenital warts.

According to some embodiments, the GcMAF present in the pharmaceutical composition comprises the amino acid sequence as set forth in any one of SEQ ID NOs:1-3 (Gc1f, Gc1s and Gc2, respectively), or a biologically active fragment thereof, or a combination thereof. Each possibility represents a separate embodiment of the invention. According to additional embodiments, the GcMAF present in the pharmaceutical composition consists of the amino acid sequence as set forth in any one of SEQ ID NOs:1-3 or a combination thereof. Each possibility represents a separate embodiment of the invention.

According to further embodiments, the GcMAF fragment present in the pharmaceutical composition comprises an amino acid sequence corresponding to amino acids 400-435 of the Gc Protein. According to certain embodiments, the GcMAF fragment consists of the amino acid sequence as set forth in SEQ ID NO:4 or SEQ ID NO:5. Each possibility represents a separate embodiment of the invention.

According to additional embodiments, the pharmaceutical composition for use in treating benign or precancerous papilloma is formulated for parenteral or oral administration. According to further embodiments, the pharmaceutical composition is formulated for subcutaneous, intramuscular, intradermal, or intravenous administration. Each possibility represents a separate embodiment of the invention. According to a certain embodiment, the pharmaceutical composition is formulated for subcutaneous or intramuscular administration.

According to some embodiments, the pharmaceutical composition for use in treating benign or precancerous papilloma is formulated in a form selected from the group consisting of a solution, an emulsion, a suspension, powder, a tablet, and a capsule. Each possibility represents a separate embodiment of the invention. According to a certain embodiment, the pharmaceutical composition is formulated as a solution suitable for subcutaneous or intramuscular administration.

According to further embodiments, the pharmaceutical composition for use in treating benign or precancerous papilloma is formulated for subcutaneous or intramuscular administration and comprises phosphate buffer, sodium chloride and polysorbate 80, wherein the pharmaceutically acceptable carrier is water. According to a certain embodiment, the pharmaceutical composition comprises phosphate buffered saline and 0.005% (w/w) polysorbate 80.

According to some embodiments, the pharmaceutical composition for use in treating benign or precancerous papilloma, if administered subcutaneously or intramuscularly, is administered once a week for at least two weeks. Alternatively the pharmaceutical composition being administered for a period of two weeks up to twelve months or for any integer in between. Each possibility represents a separate embodiment of the invention.

According to some embodiments, if the pharmaceutical composition being administered parenterally, e.g., subcutaneously or intramuscularly, the dosage of GcMAF ranges from about 10 pg/kg to about 200 μg/kg of the subject's weight. According to some embodiments, the dosage of GcMAF ranges from about 100 pg/kg to about 100 ng/kg of the subject's weight. According to yet further embodiments, the dosage of GcMAF ranges from about 0.5 ng/kg to about 20 ng/kg of the subject's weight. According to further embodiments, the dosage of GcMAF ranges from about 1 ng/kg to about 10 ng/kg of the subject's weight.

According to a certain embodiment, the pharmaceutical composition for use in treating JRRP comprises GcMAF which consists of the amino acid sequence as set forth in any one of SEQ ID NOs: 1-3, the pharmaceutical composition being administered subcutaneously once a week for a period of at least two weeks, and the dosage of GcMAF ranges from about 100 pg/kg to about 100 ng/kg body weight.

According to additional embodiment, the oral papilloma is canine oral papillomatosis.

According to another aspect, the present invention provides a pharmaceutical composition comprising GcMAF, or a biologically active fragment thereof, and a pharmaceutically acceptable carrier for use in preventing recurrent respiratory papillomatosis (RRP) according to the principles of the present invention. According a certain embodiment, the RRP is Juvenile RRP.

According to another aspect, the present invention provides a method for treating benign or precancerous papilloma comprising administering to a subject in need of such treatment a pharmaceutical composition comprising a therapeutically effective amount of GcMAF, or a biologically active fragment thereof, and a pharmaceutically acceptable carrier according to the principles of the present invention.

Other objects, features and advantages of the present invention will become clear from the following description and drawings.

### BRIEF DESCRIPTION OF THE FIGURES

FIGS. 1A-B show photographs of a dog afflicted with severe canine papillomatosis. The photographs show lesions on the face (FIG. 1A) and in the mouth (FIG. 1B) of the untreated dog.

FIGS. 2A-B show the effect of GcMAF treatment on the dog afflicted with severe canine papillomatosis of FIG. 1. The photographs show lesions on the face (FIG. 2A) and in the mouth (FIG. 2B) of the dog after 12 weekly injections of GcMAF.

FIGS. 3A-B show the effect of GcMAF treatment on the dog afflicted with severe canine papillomatosis of FIG. 1. The photographs show lesions on the face (FIG. 3A) and in the mouth (FIG. 3B) of the dog after 15 weekly injections of GcMAF.

FIGS. 4A-B show the effect of GcMAF treatment on the dog afflicted with canine papillomatosis of FIG. 1. The photographs show no lesions on the face (FIG. 4A) and in the mouth (FIG. 4B) of the dog after 22 weekly injections of GcMAF.

FIGS. 5A-D show a dog afflicted with moderate canine papillomatosis. The photographs show lesions on the face (FIGS. 5A and 5C) and in the mouth (FIGS. 5B and 5D) of the untreated dog.

FIGS. 6A-D show the effect of GcMAF treatment on the dog afflicted with moderate canine papillomatosis of FIG. 5. The photographs show the disappearance of the lesions from the mouth (FIGS. 6A and 6B) and from the face (FIGS. 6C and 6D) of the dog after 8 weekly injections of GcMAF.

FIGS. 7A-C show a dog afflicted with moderate canine papillomatosis. The photographs show lesions in the mouth of the untreated dog (FIGS. 7A-C).

FIGS. 8A-C show the effect of GcMAF treatment on the dog afflicted with moderate canine papillomatosis of FIG. 7. The photographs show the disappearance of the lesions from the mouth of the dog after 14 weekly injections of GcMAF (FIGS. 8A-C).

### DETAILED DESCRIPTION OF THE INVENTION

The present invention provides pharmaceutical compositions comprising GcMAF or a biologically active fragment thereof for use in treating benign or precancerous papillomas.

The inventors of the present invention disclose, for the first time, that GcMAF is a highly effective medicament for moderate and severe canine papillomatosis. A series of 3 to 22 subcutaneous injections of GcMAF totally eliminated the papilloma lesions from the mouth and from the face of the treated dogs. The healing effect was long-lasting and the disease did not recur. Thus, the GcMAF is shown to be an efficacious medication for treating benign or precancerous papillomas.

### GcMAF

Gc protein, also designated vitamin D binding protein (DBP), is a plasma protein of an apparent molecular weight of 52 kDa with specific oligosaccharides attached thereto. Gc protein can be purified from blood serum or plasma by any method known to a person skilled in the art. Gc protein of high purity can be isolated from serum or plasma by 25-hydroxyvitamin D₃-Sepharose affinity chromatography. The Gc protein can also be purified by actin-agarose affinity chromatography which takes advantage of the binding specificity of the Gc protein for actin. Previous publications disclose the use of glycosidases: β-galactosidase in combination with sialidase and/or mannosidase to produce Gc derived macrophage activating factor (GcMAF) preparations (see, for example, U.S. Pat. No. 5,177,002 and WO 2012/137199, the content of which is incorporated by reference as if fully set forth herein).

Alternatively, the Gc protein can be obtained from isolated cDNA encoding the Gc protein or the Gc protein small domain (domain III). Cloning and expression of the Gc protein and the Gc domain III was described in U.S. Pat. No. 6,410,269. The method described therein employs a human liver cDNA library in bacteriophage λgt11 (Clontech, Palo Alto, Calif.) for isolating a full length cDNA encoding the human Gc protein, and the use of the baculoviral expression system in insect cells for the protein expression. However, mammalian cell systems are preferred for expressing a cDNA encoding the Gc protein or active fragment thereof. Preferably, expression is performed in eukaryotic cells so that the Gc protein or its active domain is correctly glycosylated. Any such cell system known in the art can be used, for example Chinese hamster ovary (CHO) cells, BHK cells, human embryonic kidney HEK293 cells and Saccharomyces cerevisiae. Accordingly, any eukaryotic expression vector can be used, including, but not limited to, pCI-NEO, pWE3, pcDNA3.1 and pCM182. Insertion of the vector into the selected cell system can be performed, for example, by electroporation, by lipid transfection such as TransFectin or by any chemical method known to a person skilled in art, with or without amplification. The transfection may result in transient or stable expression, both forms being adequate to obtain the desired Gc protein or fragment thereof. The expressed protein, being the precursor of active MAF, can then be extracted from the cells or collected from the growth media by any method known in the art.

Gc protein is a polymorphic protein which appears in two major phenotypes as demonstrable by gel electrophoresis analysis: Gc1 and Gc2. The entire nucleotide coding sequences of the Gc1 and Gc2 genes and the predicted amino acid sequences have been reported (Cook et al., 1985. J. Clin. Invest. 76: 2420; Yang et al., 1985. Proc. Natl. Acad. Sci. USA 82 7994). Gc1 is further divided into Gc1f and Gc1s subtypes, which migrate electrophoretically as two bands (“fast” and “slow”), due to a variation in one amino acid residue.

Gc1 protein is the major subtype of human Gc protein. It carries a trisaccharide composed of N-acetylgalactosamine (GalNAc) attached to the core protein with sialic acid and galactose (in Gc1f) or mannose and galactose (in Gc1s); galactose is believed to be the outer oligosaccharide moiety. Gc2 has a simple glycosylation pattern with a core GalNAc linked to a terminal galactose moiety. Gc1f and Gc1 s* oligosaccharides can be hydrolyzed in vitro by β-galactosidase, preferably immobilized β-galactosidase such as to acrylic beads, to yield a macrophage pro-activating factor, which in turn can be hydrolyzed by sialidase (also known as neuraminidase), preferably immobilized sialidase, to yield a macrophage activating factor derived from the Gc protein denoted GcMAF. Animal, e.g., mouse or dog, DBP carries a disaccharide composed of N-acetylgalactosamine with a terminal galactose, similarly to that of Gc2. Hydrolysis of this disaccharide by β-galactosidase also generates a potent MAF denoted GcMAF.

The terms “Gc protein” or “vitamin D-binding protein” as used herein refer to human or animal Gc protein, to all genotypes and polymorphic forms, including glycosylation forms, e.g., Gel, Gc2, Gc1f, Gc1s and Gc1s*, and biologically active variants and fragments thereof. The term “biologically active” variant or fragment as used herein refers to any variant or fragment of Gc protein which upon deglycosylation produces a GcMAF variant or fragment, the GcMAF variant or fragment thus produced is capable of activating macrophages and has an N-acetylgalactosamine group linked to the core protein, most likely to a threonine residue.

The term “macrophage activating capability” as used herein refers to the ability of GcMAF, fragments and variants thereof to induce cytotoxicity or phagocytosis of a target cell by macrophages and/or to induce cytokine or chemokine release from macrophages and/or to induce Fc receptor expression or translocation. Macrophage activating capability also encompasses the ability of GcMAF, fragments and variants thereof to induce or to enhance dendritic cell activity, e.g., langerhans cell activity, so that the antigen presenting function of these cells is higher than in the absence of GcMAFs. Each of the activities, alone or in combination, enhances the cell killing or cytotoxic effect mediated by macrophages.

The term “GcMAF” refers to Gc protein which has been subjected to deglycosylation with pβ-galactosidase alone or in combination with sialidase and/or mannosidase. According to one embodiment, GcMAF is produced by stepwise in vitro deglycosylation of isolated human Gc protein with pβ-galactosidase followed by sialidase. The GcMAF is therefore deglycosylated as compared to the untreated glycosylated Gc protein, yet it still has the N-acetylgalactosamine group linked to the core protein, most likely to a threonine residue.

According to some embodiments, the GcMAF comprises the amino acid sequence as set forth in SEQ ID NO:1, SEQ ID NO:2 or SEQ ID NO:3, having a GalNAc moiety linked to a threonine residue at position 418 or 420 of the Gc protein. Each possibility represents a separate embodiment of the present invention. According to certain embodiments, GcMAF consists of the amino acid sequence selected from the group consisting of: SEQ ID NO:1, SEQ ID NO:2 and SEQ ID NO:3. Each possibility represents a separate embodiment of the present invention.

The term “fragment” as used herein refers to any portion of the full length amino acid sequence of Gc protein which has less amino acids than the full length amino acid sequence of Gc protein, e.g., less than the 458 amino acids of Gc proteins of SEQ ID NOs:1 to 3, which portion has N-acetylgalactosamine group linked to an amino acid residue, typically to a threonine, and maintains macrophage activating capability. Typically, a portion of a full length protein is a peptide or a polypeptide. By “peptide” it is meant an amino acid sequence consisting of not more than 50 amino acids. By “polypeptide” it is meant an amino acid sequence generally consisting of more than 50 amino acid residues, typically up to 200 amino acid residues.

According to some embodiments, the Gc protein fragment comprises an amino acid sequence corresponding to amino acids 400-435 of Gc Protein. Alternatively, the Gc fragment comprises the Gc protein domain III corresponding to amino acids 375-458 of the mature protein. The Gc fragment Domain III, corresponding to amino acids 375-458 of the mature Gc protein, consists of the amino acid sequence as set forth in either SEQ ID NO:4 or SEQ ID NO:5. The N-acetylgalactosamine in these amino acid sequences is linked to threonine at positions 44 or 46.

According to some embodiments, GcMAF variants or fragments retain the desired biological activities of the native GcMAF such that the variants or fragments have the same therapeutic effect as of the native GcMAF when administered to a subject. That is, according to some embodiments, the variant polypeptide will serve as a therapeutically active agent in a pharmaceutical composition in a manner similar to that observed for the native polypeptide. As used herein, “variant” relates to either the native Gc protein or a fragment of the native Gc protein comprising one or more amino acid substitutions, insertions, or deletions, and or other modifications such as glycosylation.

According to some embodiments, biologically active variants of Gc protein or Gc protein fragments have at least 50%, preferably at least 60%, more preferably at least 70%, 80%, 90%, 95%, and most preferably about 98% amino acid sequence identity to the corresponding full length amino acid sequence of the native Gc protein or fragment thereof, respectively, which serve as the basis for comparison. Each possibility represents a separate embodiment of the present invention. According to some embodiments, the biologically active variants have at least 70%, 80%, 90%, 95%, and most preferably about 98% amino acid sequence identity to Domain III of the Gc protein, which serves as the basis for comparison. Each possibility represents a separate embodiment of the present invention. As used herein, the term “sequence identity” relates to having the same amino acid residues within a variant polypeptide and the corresponding polypeptide molecule that serves as a reference when a specified, contiguous segment of the amino acid sequence of the variant is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

When considering percentage of amino acid sequence identity, some amino acid residues may differ as a result of conservative amino acid substitutions, which do not affect the properties of the protein function. In these instances, percent sequence identity may be adjusted upwards to account for the similarity in conservatively substituted amino acids. Thus, substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

### Pharmaceutical Compositions

The present invention provides pharmaceutical compositions comprising a therapeutically effective amount of macrophage activating factor derived from Gc protein denoted GcMAF or a biologically active fragment or variant thereof, and a pharmaceutically acceptable carrier or diluent, for use in treating or preventing benign or precancerous papillomas.

The term “pharmaceutical composition” as used herein refers to a composition comprising at least one pharmaceutically active ingredient.

The pharmaceutical compositions of the present invention comprise a pharmaceutically acceptable carrier. The term “carrier” refers to a diluent or vehicle with which the therapeutic compound is administered. Carrier(s) are “acceptable” in the sense of being compatible with the other ingredients of the composition and not deleterious to the recipient thereof. Such pharmaceutical carriers can be sterile liquids, such as water; oils, including those of petroleum, animal, vegetable or synthetic origin, such as peanut oil, soybean oil, mineral oil, sesame oil and the like; polyethylene glycols; glycerin; propylene glycol; or other synthetic solvents. For injectable solutions, water is a preferred carrier when the pharmaceutical composition is administered intravenously. Saline solutions and aqueous dextrose and glycerol solutions can also be employed as liquid carriers for injectable solutions.

The pharmaceutical composition comprising GcMAF can further comprise agents for adjustment of tonicity including, but not limited to, sodium chloride or dextrose. According to some embodiments, the pharmaceutical composition of the present invention comprises sodium chloride in order to keep the osmotic pressure of composition suitable for an injectable preparation. The amount of sodium chloride ranges from about 25 mM to about 300 mM.

The pharmaceutical composition comprising GcMAF can further comprise a buffering agent. Buffering agents that can be included in the compositions of the present invention are, for example, phosphate buffer, acetate buffer, or citrate buffer. According to some embodiments, injectable solutions of the invention can be formulated in physiologically compatible buffers such as Hank's solution, Ringer's solution, or physiological salt buffer.

The pharmaceutical composition can further comprise a surfactant. According to some embodiments, the surfactant is a nonionic surfactant. Nonionic surfactants include, but are not limited to, sorbitan fatty acid esters, polyoxysorbitan fatty acid esters, polyoxyalkylene higher alcohol ethers, and polyoxyalkylene higher alcohol esters. Thus, nonionic surfactants include polyoxyethylene sorbitol esters such as polysorbate 80 (TWEEN® 80), polysorbate 60 (TWEEN® 60) and polysorbate 20 (TWEEN® 20), Tyloxapol; polyoxyethylene isooctylphenyl ethers such as Triton X-100, polyoxyethylene nonylphenyl ethers such as NP-40, polyoxyethylene dodecyl ethers such as Brij 58, octyl glucoside, and alkyl maltoside such as n-dodecyl-beta-D-maltoside; Poloxamer 4070; Poloxamer 188; and polyoxyl 40 stearate. Each possibility is a separate embodiment of the invention. TWEEN® and Poloxamer surfactants are preferred because they are FDA approved for human use.

According to some embodiments, the pharmaceutical composition comprises GcMAF, a nonionic surfactant, a tonicity agent, a buffering agent, and water (see WO 2014/199373 to the applicant of the present invention, the content of which is incorporated as if fully set forth herein). According to certain embodiments, the pharmaceutical composition comprises GcMAF, polysorbate 80, sodium chloride, phosphate buffer, and water.

The pharmaceutical compositions of the present invention can be formulated as a liquid. The liquid composition can be stored as is or can be stored in a frozen state, or in a dried form for later reconstitution into a liquid form or other form suitable for administration to a subject.

The GcMAF or fragments thereof according to the invention may be formulated as neutral or salt forms. Pharmaceutically acceptable salts include the acid addition salts (formed with free amino groups) which are formed with inorganic acids such as hydrochloric or phosphoric acids, or with organic acids such as acetic, oxalic, tartaric and maleic. Salts formed with the free carboxyl groups may be derived from inorganic bases such as sodium, potassium, ammonium, calcium, or ferric hydroxides, or from organic bases such as isopropylamine, trimethylamine, 2-ethylamino ethanol, histidine and procaine.

The compositions may be suitably formulated for subcutaneous, intramuscular, intraperitoneal or intravenous administration and comprise sterile aqueous solutions, which are preferably isotonic. Such formulations are typically prepared by dissolving solid active ingredients in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (for example trehalose), amino acids, inorganic acids and admixtures thereof. Stabilizers are used in aqueous solutions at the appropriate concentration and pH. The pH of the pharmaceutical composition of the present invention is adjusted to be within the range of 5.0-9.0, preferably within the range of 6-8. In formulating the GcMAF or fragment thereof according to the invention, anti-adsorption agents may be used.

According to some embodiments, the preparations described herein are formulated for parenteral administration, e.g., by bolus injection or continuous infusion.

According to some embodiments, the compositions are formulated as suspensions, solutions or emulsions in oily or aqueous vehicles, and contain formulatory agents such as suspending, stabilizing and/or dispersing agents.

According to some embodiments, the compositions of the invention may be formulated for oral administration in liquid solutions, emulsions, suspensions, and the like. The pharmaceutically-acceptable carriers suitable for preparation of such compositions are well known in the art. Proteins that are orally administered need to be protected as to avoid digestion by the gastrointestinal system.

The proteins of the invention can be coated with enteric coating layer(s) as to protect them from digestion. Enteric coating layer(s) may be applied using standard coating techniques. The enteric coating materials may be dissolved or dispersed in organic or aqueous solvents and may include one or more of the following materials: methacrylic acid copolymers, shellac, hydroxypropylmethylcellulose phthalate, polyvinyl acetate phthalate, hydroxypropylmethylcellulose trimellitate, carboxymethylethylcellulose, cellulose acetate phthalate or other suitable enteric coating polymer(s). The pH at which the enteric coat will dissolve can be controlled by the polymers, combination and ratio of selected polymers, and/or their side groups. For example, dissolution characteristics of the polymer film can be altered by the ratio of free carboxyl groups to ester groups. Enteric coating layers also contain pharmaceutically acceptable plasticizers such as triethyl citrate, dibutyl phthalate, triacetin, polyethylene glycols, polysorbates or other plasticizers. Additives such as dispersants, colorants, anti-adhering and anti-foaming agents may also be included.

The compositions of the invention may be formulated as controlled release preparations which may be achieved through the use of a polymer to complex or absorb the proteins of the invention. Appropriate polymers for controlled release formulations include, for example, polyester, polyamino acids, polyvinyl, pyrrolidone, poly (lactic acid), ethylenevinylacetate, ethylene vinylacetate copolymers, and cellulose derivatives such as methylcellulose. Alternatively, it is possible to entrap the proteins of the invention in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, and nanoparticles.

The compositions of the present invention can further comprise an additional therapeutic agent. According to certain embodiments, the therapeutic agent is an antiviral drug.

### Therapeutic Uses of GcMAF

The present invention provides uses of the pharmaceutical compositions comprising a therapeutically effective amount of GcMAF for treating papilloma virus-induced benign or precancerous papillomas.

Infection with human papilloma virus (HPV) is recognized as one of the major causes of infection-related cancer worldwide. However, papilloma viruses also cause benign or precancerous papillomas in a wide variety of mammals. The papillomas can be found in the passages of the respiratory and digestive tract, in the mouth and throat, in the anal and genital organs, and in the conjunctiva of the eye. The papillomas can also be found on the skin where they are more commonly referred to as warts.

The term “treating” as used herein refers to ameliorating symptoms associated with the disease, to lessen the severity of the disease or cure the disease. The term “treating” also refers to attenuating or preventing relapse or recurrence of the disease.

The term “therapeutically effective amount” as used herein refers to an amount of the active agent, namely GcMAF or a biologically active fragment or variant thereof that is sufficient to treat, alleviate, and/or inhibit one or more symptoms of the disease in an individual. The therapeutically effective amount will vary depending on the active agent, formulation, the disease and its severity and the age, weight, physical condition and responsiveness of the subject to be treated.

The term “precancerous” is intended to mean the presence or development of abnormal, premalignant cells. Precancerous papillomas are typically characterized by unregulated cell growth or dysplasia that will progress to cancer.

The benign or precancerous papillomas that can be treated by the pharmaceutical composition comprising GcMAF of the present invention include, but are not limited to, papillomas of the larynx known as Respiratory Papillomatosis or Recurrent Respiratory Papillomatosis, oral or buccal papillomas known as oral papillomatosis, papillomas of the lip, the tongue, the pharynx, the esophagus, the cervix, the vagina, the anus, the penis, and papillomas of the skin or warts.

Recurrent respiratory papillomatosis (RRP) is caused by HPV subtypes 6 and 11 and is characterized by the outgrowth of benign warty lesions in the larynx and vocal cords. RRP can affect both children and adults, with young children having a more severe disease course due primarily to narrow airways which are easily obstructed by lesions. The disease can therefore be a life-threatening condition, primarily for young children. Surgical debulking of lesions to avoid respiratory distress is the current standard of care, and in children RRP necessitates multiple, frequent surgical interventions.

Assessment of the severity of RRP in children is based on the number of surgical procedures the pediatric patient has undergone during the past 12 months as follows:

Mild—less than 4 surgeries per year;
Moderate—4-8 surgeries per year;
Aggressive/severe—more than 8 surgeries per year.

(see, for example, Derkay et al., 2004, International Journal of Pediatric Otorhinolaryngology 68: 1493-1498).

Adult-onset RRP is typically diagnosed at 20-40 years of age. In adult patients, the number of surgeries is lower than in children and the severity of the disease is established as follows:

Mild or moderate—less than 2 surgeries per year;
Aggressive/severe—more than 2 surgeries per year.

Effectiveness of GcMAF in treating RRP can be determined by assessment of lesion or papilloma size and/or number by nasopharyngoscopy or during debulking surgery procedures. Thus, GcMAF can reduce the size and/or number of the papillomas by at least 20%, 30%, 40%, 50%, 60%, 70%, 80% 90% or can eradicate the papillomas. Each possibility is a separate embodiment of the invention. Effectiveness of GcMAF in treating RRP can also be assessed by increasing the interval between surgeries and/or by delaying disease recurrence.

It should be noted that the benign or precancerous papillomas treated by GcMAF according to the principles of the present invention can be induced by human papilloma virus (HPV) as well as by non-human papilloma viruses. For example, oral papillomatosis that can be treated by GcMAF is canine oral papillomatosis as exemplified herein below.

The pharmaceutical composition of the invention may be administered by any suitable administration route, such as by parenteral or by oral administration route. According to some embodiments, the route of administration is via parenteral injection. According to additional embodiments, the parenteral route of administration is selected from the group consisting of subcutaneous, intramuscular, intradermal, intraperitoneal, intravenous, intraarterial, and intrathecal. The compositions of the invention can be administered locally.

The pharmaceutical composition of the present invention can be administered once a week, twice a week, three times a week for a period of at least two weeks, three weeks, four weeks, 2 months, 3 months, 4 months, 5, 6, or 12 months or any integer in between as required so as to reduce the number and/or size of the papillomas or to eradicate the papillomas. Each possibility represents a separate embodiment of the invention. According to some embodiments, the pharmaceutical composition comprising GcMAF is administered once a week for duration sufficient to eradicate the papillomas and to prevent recurrence.

According to some embodiments, the pharmaceutical composition is administered once in two weeks or once a month for at least one year after the papilloma is eradicated, thereby preventing the recurrence of the benign or precancerous papilloma. According to additional embodiments, the pharmaceutical composition is administered once in two weeks, or once a month for the rest of the subject's life, thereby preventing the recurrence of the benign or precancerous papilloma. Each possibility represents a separate embodiment of the invention.

The dosage of GcMAF administered can range from about 10 pg/kg to about 200 μg/kg of the subject's weight or any integer in between. According to some embodiments, the dosage of GcMAF administered ranges from about 0.5 ng/kg to about 100 ng/kg of the subject's weight. According to other embodiments, GcMAF is administered in a dosage ranging from about 1 ng/kg to about 10 ng/kg of the subject's weight. According to yet other embodiments, GcMAF is administered in a dosage of about 0.1, 0.2, 0.4, 1, 1.5, 2, 2.5, 5, 10, 20, 30, 40, 100, 500, 1000 ng/kg of the subject's weight and any integer there between. Each possibility represents a separate embodiment of the invention.

The term “about” in reference to a numerical value stated herein is to be understood as the stated value +/−10%.

The pharmaceutical composition can also be delivered by slow-release delivery systems, pumps, and other known delivery systems for continuous infusion. Dosing regimens may be varied to provide the desired circulating levels of GcMAF or a biologically active fragment or variant thereof based on pharmacokinetics. Thus, doses are calculated so that the desired circulating level of a therapeutic agent is maintained.

The pharmaceutical compositions of the present invention are also suitable for prophylactic treatment, for inhibiting or attenuating the recurrence of benign or precancerous papillomas whether removed by the pharmaceutical composition of the present invention or by surgery. For example, a combination of surgeries to remove lesions in RRP patients and GcMAF administration can be useful to elongate the intervals between surgeries until no surgery is required.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### Example 1

### Effect of GcMAF on Severe Canine Papillomatosis

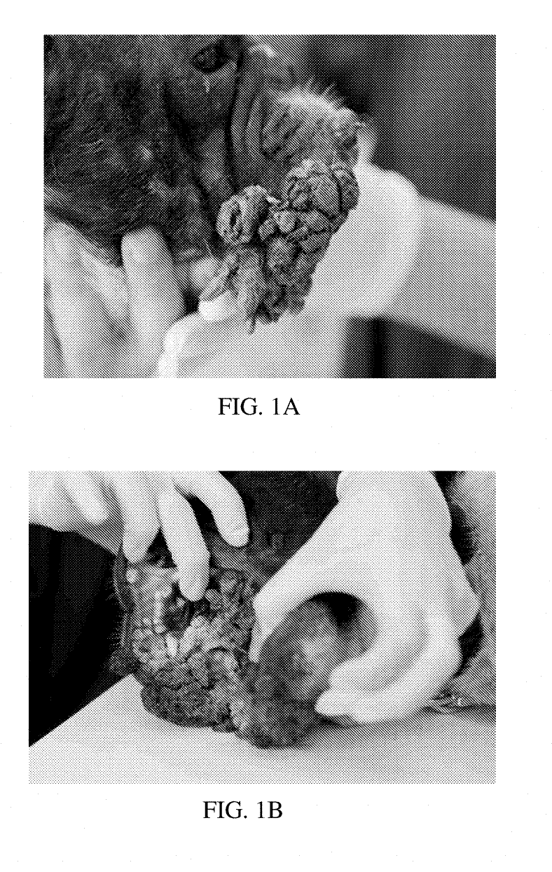

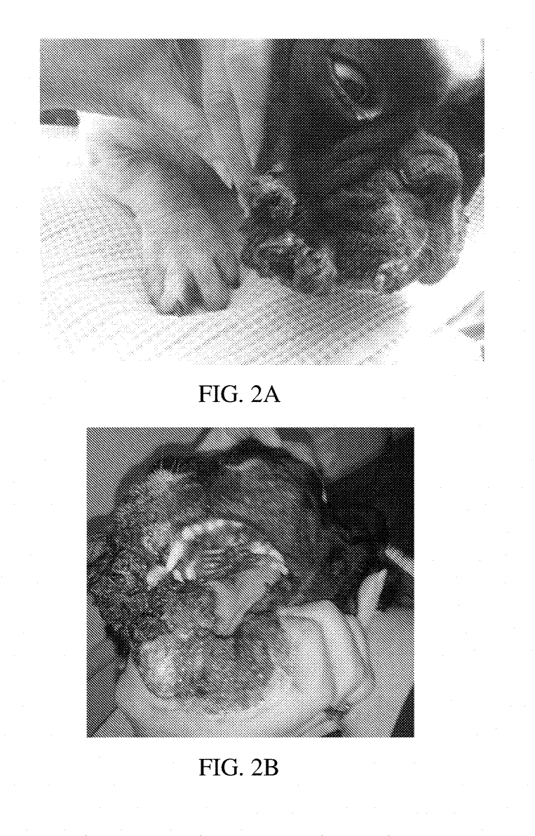

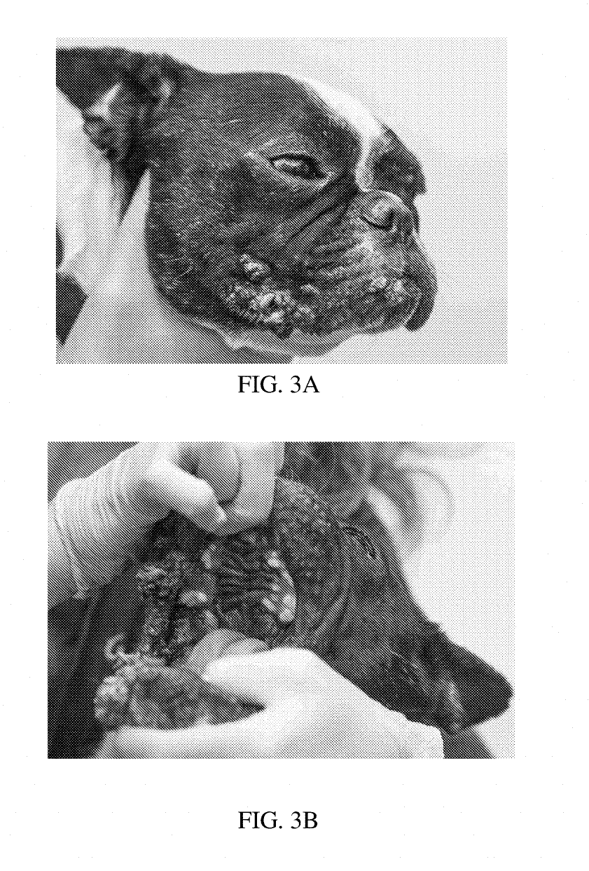

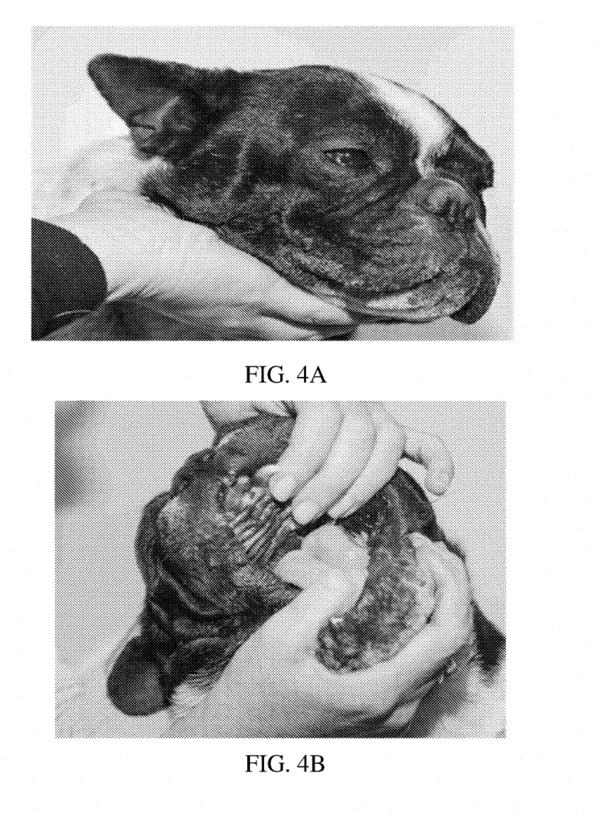

A five year old dog afflicted with severe canine papillomatosis, having papilloma lesions or warts on its face and in the mouth (FIGS. 1A and 1B), stopped eating solid food and lost weight. At this stage, none of the available medicaments was successful in eradicating the warts, and therefore GcMAF was administered to the dog, aiming at improving its clinical condition. For that end, the dog was treated with weekly subcutaneous (SC) injections of GcMAF (100 ng/70 kg) prepared in phosphate buffered saline and 0.005% Tween-80. At the first week of treatment, the disease progression was arrested and no additional lesions appeared. After the second injection of GcMAF, the dog started to eat solid food and to gain weight. In the following weeks the dog's condition continued to improve and after 12 injections nearly all the lesions inside the mouth disappeared and the lesions on the face were significantly reduced (FIGS. 2A and 2B). After 15 weekly injections of GcMAF, the dog's mouth was clear of lesions and only few minor lesions were left on the face (FIGS. 3A and 3B). After 21 injections of GcMAF, the dog showed a complete recovery and no lesions in the mouth or on the face were seen (FIGS. 4A and 4B).

### Example 2

### Effect of GcMAF on Moderate Canine Papillomatosis

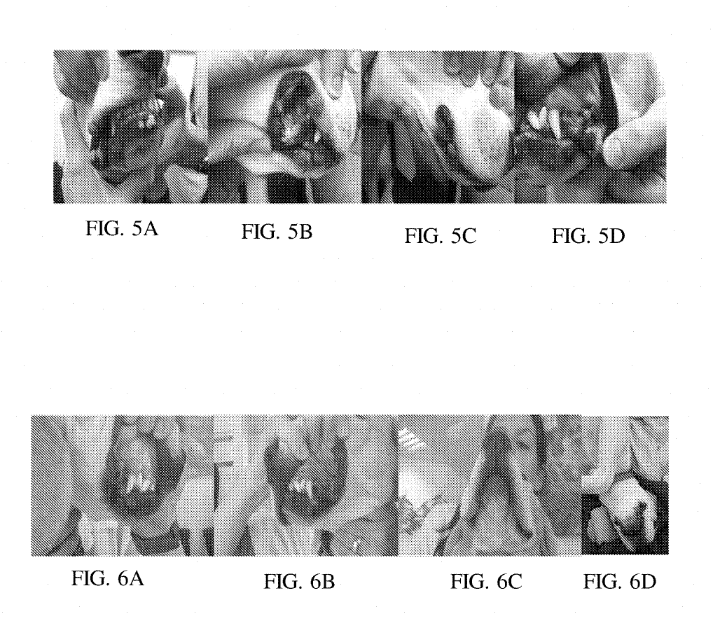

A one year old dog afflicted with moderate canine papillomatosis, having papilloma lesions on its face and in the mouth (FIG. 5A-D), was treated with GcMAF aiming at improving its clinical condition. For that end, the dog received weekly subcutaneous (SC) injections of GcMAF (100 ng/70 kg) in an aqueous solution containing phosphate buffered saline containing 0.005% Tween 80. After 3 injections of GcMAF, the dog showed a complete recovery and no lesions in the mouth or on the face were seen. The dog received 5 additional weekly injections after the lesions disappeared (FIGS. 6A-D).

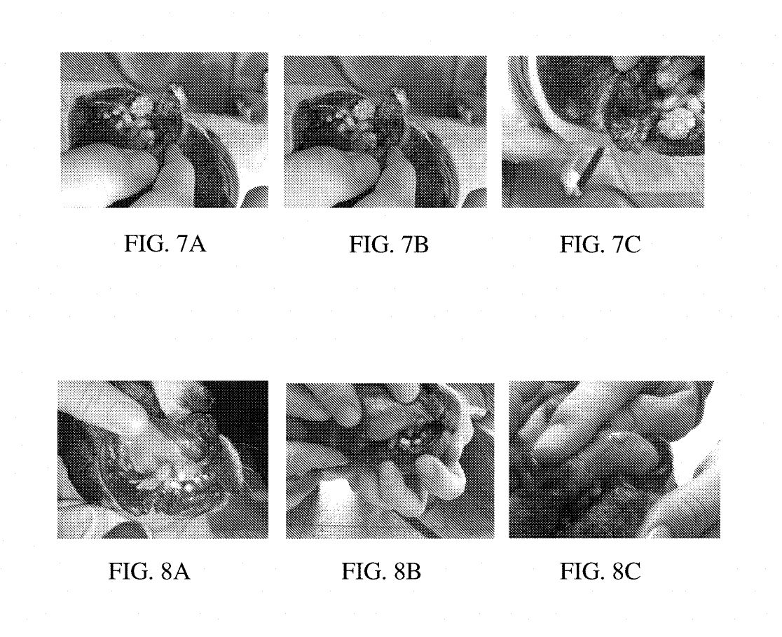

Another dog (15 years old) afflicted with moderate canine papillomatosis, having papilloma lesions in the mouth (FIGS. 7A-C), was treated with GcMAF aiming at improving its clinical condition. For that end, the dog received weekly subcutaneous (SC) injections of GcMAF (100 ng/70 kg) in phosphate buffered saline containing 0.005% Tween 80. Following 14 injections of GcMAF, the dog showed a complete recovery and no lesion were detected in the mouth (FIGS. 8A-C).

### Example 4

### Effect of GcMAF on Juvenile or Adult-Onset RRP—Clinical Study

The effect of GcMAF administered to pediatric or adult subjects is next evaluated.

Children at the age of 2 to 18 weighing 14 kg and above who have juvenile RRP (JRRP) and have undergone at least three debulking procedures during the past twelve months are enrolled to the study. These children are assessed as having moderate to severe/aggressive JRRP. Each child is injected subcutaneously once a week with phosphate buffered saline containing 0.005% Tween 80 and GcMAF (200 ng/ml), the dosage of GcMAF injected is 1.5 ng/kg body weight. The weekly injections continue up to one year, according to the physician's discretion. The child's response to treatment is assessed monthly by nasopharyngoscopy to evaluate lesion size and/or number and is compared to the lesion size and/or number at day 1 of the study, i.e., baseline reference (prior treatment). If surgery is performed, lesion size and/or number is evaluated during the debulking surgery procedure. Response assessment is also evaluated by symptomatic assessment, namely assessment of the narrowing of airways (noisy breathing/stridor) and voice hoarseness.

The study of adults having RRP includes patients of the age of 18 years and above which have undergone at least two debulking procedures during the last twelve months. Each patient is injected subcutaneously or intramuscularly with the aqueous solution of GcMAF described herein above. The dosage of GcMAF injected is either 1.5 ng/kg or 4.5 ng/kg body weight, once a week for a period of up to twelve months, according the physician discretion. Response assessment is performed as described above for pediatric patients.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-25. (canceled)

26. A method of treating benign or precancerous papilloma comprising administering to a subject in need of such treatment a pharmaceutical composition comprising a therapeutically effective amount of a macrophage activating factor derived from Gc protein (GcMAF), or a biologically active fragment thereof, and a pharmaceutically acceptable carrier.

27. The method according to claim 26, wherein the benign or precancerous papilloma is selected from the group consisting of laryngeal papilloma (recurrent respiratory papillomatosis), oral papilloma, conjuctival papilloma, verrucae plantares, verrucae vulgaris, anogenital warts, and focal epithelial hyperplasia.

28. The method according to claim 27, wherein the benign or precancerous papilloma is laryngeal papilloma (recurrent respiratory papillomatosis; RRP).

29. The method according to claim 28, wherein the RRP is juvenile RRP (JRRP).

30. The method according to claim 29, wherein the JRRP is selected from the group consisting of moderate JRRP and aggressive JRRP.

31. The method according to claim 30, wherein the JRRP is aggressive JRRP.

32. The method according to claim 28, wherein the RRP is adult-onset RRP.

33. The method according to claim 32, wherein the adult-onset RRP is aggressive adult-onset RRP.

34. The method according to claim 26, wherein GcMAF comprises the amino acid sequence as set forth in any one of SEQ ID NOs:1-3 or a biologically active fragment thereof.

35. The method according to claim 34, wherein GcMAF consists of the amino acid sequence as set forth in any one of SEQ ID NOs:1-3.

36. The method according to claim 26, wherein the pharmaceutical composition being formulated for parenteral administration.

37. The method according to claim 36, wherein parenteral administration is subcutaneous administration or intramuscular administration.

38. The method according to claim 37, wherein the pharmaceutical composition comprises a pharmaceutically acceptable carrier which is water, further comprising phosphate buffer, sodium chloride and polysorbate 80.

39. The method according to claim 38, wherein the pharmaceutical composition comprises phosphate buffered saline and 0.005% (w/w) polysorbate 80.

40. The method according to claim 36, wherein the pharmaceutical composition being administered weekly for at least two weeks or for a period of up to twelve months.

41. The method according to claim 40, wherein GcMAF being administered in a dosage of 10 pg/kg to 200 □g/kg of the subject's body weight.

42. The method according to claim 26, wherein the benign or precancerous papilloma is JRRP, GcMAF consists of the amino acid sequence as set forth in any one of SEQ ID NOs:1-3, the pharmaceutical composition being administered subcutaneously once a week for a period of at least two weeks, and wherein the dosage of GcMAF ranges from about 100 pg/kg to about 100 ng/kg of the subject's body weight.

43. The method according to claim 27, wherein the oral papilloma is canine oral papillomatosis.

44. A method of preventing recurrent respiratory papillomatosis (RRP) comprising administering to a subject in need of such treatment a pharmaceutical composition comprising a therapeutically effective amount of a macrophage activating factor derived from Gc protein (GcMAF), or a biologically active fragment thereof, and a pharmaceutically acceptable carrier.

45. The method according to claim 44, wherein the RRP is juvenile RRP (JRRP).
